(19) United States
(12) Patent Application Publication
(10) Pub. No.: US 2019/0224491 A1
(43) Pub. Date: Jul. 25, 2019

(54) PERIPHERALLY FOCUSED LIGHT FROM WEARABLE AUDIO INTERFACES

(71) Applicant: BOSE CORPORATION, Framingham, MA (US)

(72) Inventor: Andrew D. Dominijanni, Newton, MA (US)

(21) Appl. No.: 16/247,694

(22) Filed: Jan. 15, 2019

Related U.S. Application Data
(60) Provisional application No. 62/619,240, filed on Jan. 19, 2018.

(51) Int. Cl. A61N 5/06; H04R 1/10

(52) U.S. Cl. CPC A61N 5/06; H04R 1/1058; A61N 2005/0652; A61N 2005/0632; H04R 1/1008

(57) ABSTRACT

Audio systems and methods are provided that include an acoustic driver and an optical light source, each coupled to a head-worn structure. The acoustic driver is configured to convert an audio signal into an acoustic signal in proximity to an ear of the user. The optical light source is configured to direct optical energy toward an eye of the user. A user interface coupled to the light source is configured to control at least one of a color selection, a luminance, or duration of the optical energy.

### CROSS-REFERENCE TO RELATED APPLICATIONS

This application claims priority under 35 U.S.C. § 119(e) to U.S. Provisional Patent Application Ser. No. 62/619,240, titled PERIPHERALLY FOCUSED LIGHT FROM WEARABLE AUDIO INTERFACES, filed Jan. 19, 2018, which is incorporated by reference herein in its entirety for all purposes.

### BACKGROUND

Headphone systems provide audio enhancement, listening, and communications (e.g., telephone) capabilities to users. Headphone systems are often used during travel, such as in planes, trains, cars, and other vehicles, and may provide features such as noise cancellation or reduction to reduce the stress of travel. Headphone systems may provide a platform for further functions and features that may be particularly adaptable to travel and/or stress reduction, including the effects of jetlag or sleep disruption.

### SUMMARY

Aspects and examples are directed to headphone systems and methods that are capable of providing light therapy to the user. Light may be provided by certain aspects of a headphone system, e.g., in the periphery of the user's vision, and may affect the user's mood, encourage sleep or wakefulness, ease or reduce the effects of seasonal affective disorder, and/or assist in re-establishing circadian rhythms, etc.

According to one aspect, an audio device is provided that includes a structure configured to be worn on a user's head, an acoustic driver coupled to the structure and configured to convert an audio signal into an acoustic signal in proximity to an ear of the user, an optical light source coupled to the structure and configured to direct optical energy toward an eye of the user, and an interface coupled to the light source and configured to control at least one of a color selection, a luminance, or a duration of the optical energy.

Some examples include a light pipe coupled to the light source and configured to deliver the optical energy from the light source to the eye of the user.

In certain examples, the light source may include a multi-color light emitting device configurable to produce one of a plurality of colors.

Certain examples also include a sensor coupled to the interface and configured to sense an aspect of the user's physiology. The interface may be configured to control the light source based at least in part upon the aspect of the user's physiology. In some examples, aspects of the user's physiology may include a heart rate, a respiration rate, a respiration difficulty, an exertion level, a muscle activity, or a blood component level.

Some examples include an ambient light sensor, the light source being configured to adjust the optical energy based at least in part upon a signal from the ambient light sensor.

Certain examples include an adjustable component configured to allow user-selectable delivery of the optical energy to a location other than the eye of the user.

In some examples, the optical light source may be configured to provide optical energy into the eye of the user via a peripheral area of the eye of the user.

In some examples, the optical light source may be configured to provide optical energy into the eye of the user by illuminating a portion of skin near the eye of the user.

According to another aspect, a headphone system is provided that includes a structure configured to be worn on a user's head, an earpiece coupled to the structure, an acoustic driver coupled to the earpiece and configured to render an audio signal, an optical light source coupled to one of the structure or the earpiece and configured to direct optical energy toward an eye of the user, and an interface coupled to the light source and configured to control at least one of a color selection, a luminance, or a duration of the optical energy.

Some examples also include a light pipe coupled to the light source and configured to deliver the optical energy from the light source to the eye of the user.

In certain examples, the light source includes a multi-color light emitting device configurable to produce one of a plurality of colors.

Certain examples include a sensor coupled to the interface and configured to sense an aspect of the user's physiology. The interface may be configured to control the light source based at least in part upon the aspect of the user's physiology. In some examples, aspects of the user's physiology may include a heart rate, a respiration rate, a respiration difficulty, an exertion level, a muscle activity, or a blood component level.

Some examples include an ambient light sensor, the light source being configured to adjust the optical energy based at least in part upon a signal from the ambient light sensor.

Certain examples also include an adjustable component configured to allow user-selectable delivery of the optical energy to a location other than the eye of the user.

In some examples, the optical light source is configured to provide optical energy into the eye of the user via a peripheral area of the eye of the user.

In certain examples, the optical light source is configured to provide optical energy into the eye of the user by illuminating a portion of skin near the eye of the user

Still other aspects, examples, and advantages of these exemplary aspects and examples are discussed in detail below. Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

### BRIEF DESCRIPTION OF THE DRAWINGS

Various aspects of at least one example are discussed below with reference to the accompanying figures, which are not intended to be drawn to scale. The figures are included to provide illustration and a further understanding of the various aspects and examples, and are incorporated in and constitute a part of this specification, but are not intended as a definition of the limits of the invention. In the figures, identical or nearly identical components illustrated in various figures may be represented by a like numeral. For purposes of clarity, not every component may be labeled in every figure. In the figures:

FIG. 1 is a perspective view of an example headphone set;

FIG. 2 is a left-side view of an example headphone set;

FIG. 3 is a schematic diagram of an example headphone system; and

FIG. 4 is a schematic diagram of a control methodology that may be carried out with a headphone set.

### DETAILED DESCRIPTION

Aspects and examples are directed to headphone systems and methods that include a light source to provide light to the eyes of a user of a headphone system. The light source may be controlled in various ways and may advantageously affect physiology, performance, or mood of the user. The light source may be used while traveling, working, relaxing, etc. and may be used to set a mood, encourage sleep or wakefulness, ease or reduce the effects of seasonal affective disorder, and/or assist in re-establishing circadian rhythms of the user, etc.

Throughout this disclosure the terms “headset,” “headphone,” and “headphone set” are used interchangeably and refer to any wearable audio interface or device. No distinction is meant to be made by the use of one term over another unless the context clearly indicates otherwise. Additionally, aspects and examples in accord with those disclosed herein may be applied to earphone form factors (e.g., in-ear transducers, earbuds) and/or off-ear acoustic devices (e.g., devices that are designed to not contact a wearer's ears, but are worn in the vicinity of the wearer's ears, on the head or body, e.g., shoulders) and such are also contemplated by the terms “headset,” “headphone,” and “headphone set.” Accordingly, any on-ear, in-ear, over-ear, or off-ear form-factors of personal acoustic devices are intended to be included by the terms “headset,” “headphone,” and “headphone set.” The term “earpiece” is intended to include any portion of such form factors in proximity to at least one of a user's ears.

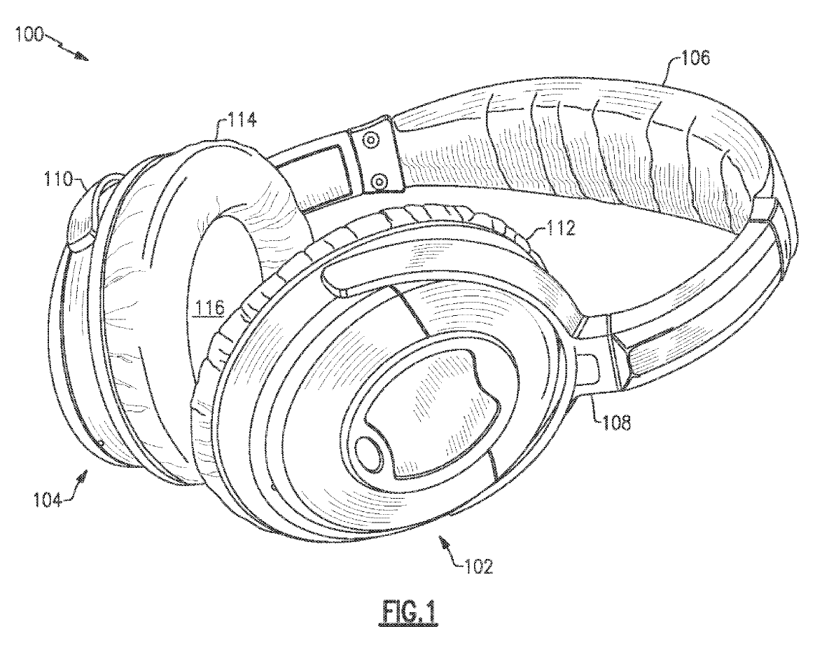

FIG. 1 illustrates one example of a headphone set. The headphones 100 include two earpieces, e.g., a right earcup 102 and a left earcup 104, coupled to a right yoke assembly 108 and a left yoke assembly 110, respectively, and intercoupled by a headband 106. The right earcup 102 and left earcup 104 include a right circumaural cushion 112 and a left circumaural cushion 114, respectively. Visible on the left earcup 104 is a left interior surface 116. While the example headphones 100 are shown with earpieces having circumaural cushions to fit around or over the ear of a user, in other examples cushions may sit on the ear, or may include earbud portions that protrude into a portion of a user's ear canal, or may include alternate physical arrangements, as discussed above. Although the example headphones 100 illustrated in FIG. 1 include two earpieces, some examples may include only a single earpiece for use on one side of the head only. Additionally, although the example headphones 100 include a headband 106, other examples may include different support structures to maintain one or more earpieces (e.g., earcups, in-ear structures, neckband, etc.) in proximity to a user's ear, e.g., an earbud may include a shape and/or materials configured to hold the earbud within a portion of a user's ear, or a personal speaker system may include a neckband to support and maintain acoustic driver(s) near the user's ears, shoulders, etc.

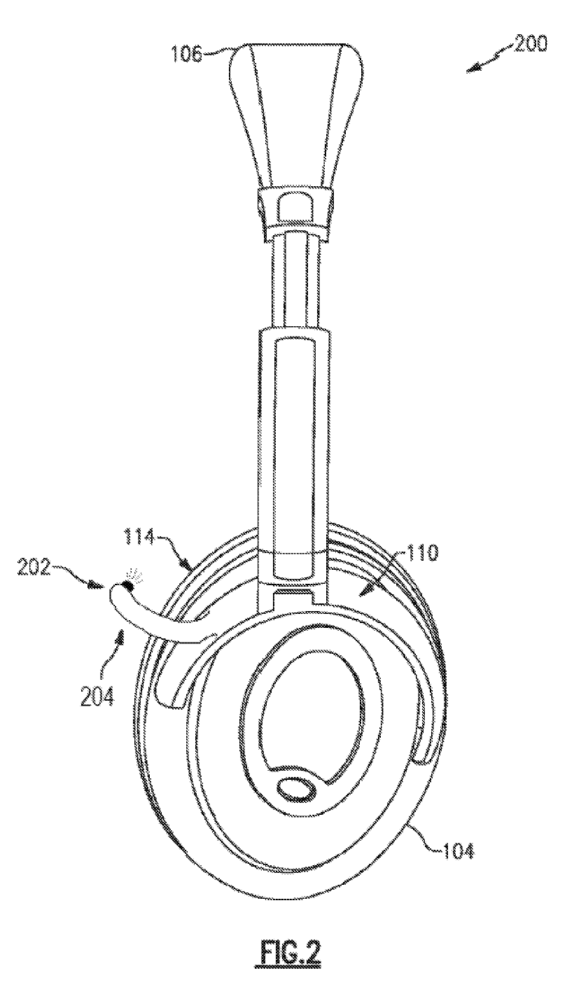

FIG. 2 illustrates another example of headphones 200 including similar structural features as the headphones 100, but also including a light source 202 positioned to be capable of producing light that reaches the user's eye or eyes. The light source 202 is associated with the left earcup 104 in this example, but may be associated with the right earcup 102 in other examples, or each of the left and right earcups may have an associated light source in yet other examples. Further, the light source 202 is shown extended from the remaining structure of the headphone 200, but in other examples a light source may be incorporated in the structure of the headphones. For example, the light source 202 may be associated with any portion of the headphones 100, and may be internal to the headphones such that the exterior form factor of the headphones may be substantially identical to that of headphones without a light source. Further, in various examples, and as described above, “headphones” as used herein may refer to any form factor of wearable audio devices, and a light source may be incorporated into the form factor or may be structurally extended away, at a mild distance, from an otherwise non-lighted form factor. In some examples, a light pipe or light guide may be provided to convey light from the light source to another position, and to direct the light in the general vicinity of one or both of the user's eyes. Protruding member 204 of FIG. 2 may be or may include a light pipe or light guide or may simply support the light source 202 on the free end of the protruding member 204.

In some examples, the light source 202 may be capable of generating monochromatic light, dichromatic light, of broad or narrow wavebands, and may be configurable or otherwise adjustable to vary a color and/or color temperature of light produced by the light source 202. In general, the light source 202 may be capable of producing light in a visible spectrum, but in some examples the light source 202 may additionally or alternately be capable of producing light in an infrared and/or ultraviolet spectrum. In some examples, the light source may be capable of generating a full visible spectrum. In certain examples, the light source may be one or more light emitting diodes (LEDs), and may include multiple LEDs of varying colors to provide a configurable range of, e.g., red, green, blue (RGB) color combinations. In some examples, the light source may include a source that emits light in one spectral range and includes a phosphor or scintillation element to convert or re-emit light energy in a different spectral range. For instance, a light source may produce ultraviolet light directed at a phosphor that absorbs the ultraviolet light and re-emits visible light.

In some examples, additional optics may be associated with the light source 202. For instance, reflectors, diffusers, lenses, optical fibers, or other light guides may be associated. In some examples, light from the light source may be collimated by various optics (e.g., lenses, parabolic or other curved reflectors) such that the light is substantially directed, to reduce or eliminate light “leakage” or “spillage” to the surroundings. Collimation may enhance the effectiveness of a light source 202 of relatively low power. For example, the light may be directed toward one or more of the user's eyes. In some examples, the light may be directed toward an alternate portion of the user's face, such as the cheek bone, eye socket bones, brow bone, bridge or sides of the nose, etc. such that the light may enter the eye as diffuse reflected light from such structures of the face. In some examples, exposure of the skin to light may have beneficial effect. In various examples, the light entering the user's eye(s) is peripheral and substantially does not interfere with the user's vision or sight of the surroundings. In some examples, the light may be diffused (independently, or in combination with other directivity or collimation) to “soften” the light reaching the user's eye(s), e.g., via a diffusive optical element and/or diffuse reflection from other facial features, etc.

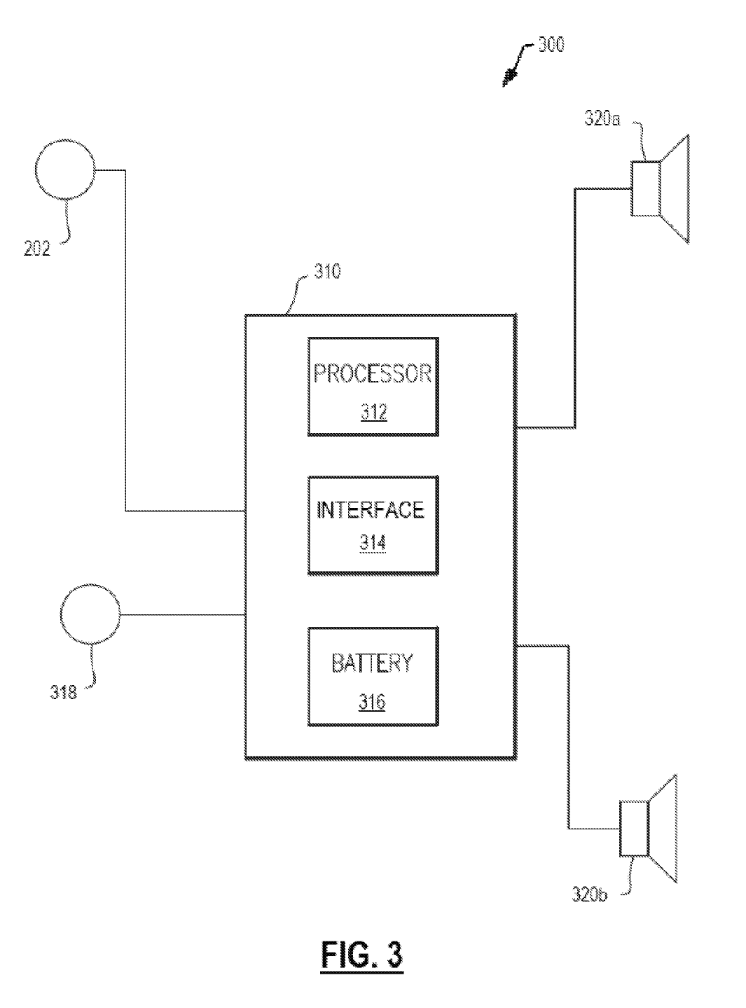

FIG. 3 is a schematic block diagram of an example headphone system 300, such as for the headphones 100, 200. The headphone system 300 includes a controller 310 that may control the light source 202 and may provide signals to acoustic drivers 320 for audio playback (e.g., right driver 320a, left driver 320b). The controller 310 includes a processor 312, and an interface 314, and may include a battery 316 and/or additional components. The interface 314, for example, may be a wired or a wireless interface, and may be configured to provide a user interface (e.g., to prompt for and/or accept feature options and selections by a user), and/or may be configured to receive program content signals for audio playback. In certain examples, the interface 314 may be a Bluetooth™ interface, or the like, for instance, that allows for streaming of audio content and user control functionality. In certain examples, the interface 314 may include components to accept an audio signal from one or more microphones (not shown) to provide content signals for playback, e.g., through the drivers 320. For example, certain headphone systems may provide amplified microphone signals to one or more driver(s) 320 for, e.g., conversational hearing assistance, enhancement of environmental sounds (birds, nature, vehicles), and the like.

The headphones 100, 200 and/or the headphone system 300 may further include one or more sensors 318, such as light sensors, and the controller 310 may receive signals from such sensors and may control the light source 202 based in part upon the sensor signals. For example, a brightness of the light source 202 may be adjusted by the controller 310 based upon the brightness of the surrounding environment, as indicated by a sensor, for example, sensor 318.

In various examples, the controller 310 may adjust the light source 202 in response to various parameters, some of which may be indicated by a sensor, for example, sensor 318. For example, sensor 318 may be or may include a microphone may detect user activity (e.g., heavy breathing, snoring, etc.) or heart rate, and the controller 310 may adjust the light source 202 in response thereto. A physiological monitor (e.g., heart rate, blood pressure, respiration, etc.) or sensor, for example, sensor 318 may provide indications of user activity or physiologic status and the controller 310 may adjust the light source 202 in response thereto. In some examples, the controller 310 may adjust the light source 202 based upon time of day. For example, a desired sleep cycle (which may be programmed or configured by the user in some examples) may control times of day when the light source 202 should be configured to produce light that is intended to aid in wakefulness and/or other times of day when the light source 202 should be configured to produce light that is intended to aid sleep.

In various examples, the light source 202 and/or associated optics may be physically adjustable. In some examples, the user may adjust the light source so that it is directed to a correct one of various locations, as discussed above. In some examples, the user may manually make physical adjustments. In some examples, physical adjustability may be mechanized, e.g., motorized, and the user may control the physical adjustment via a user interface or semi-automated control. In some examples, physical adjustability may be automated, e.g., via the controller 310, to make adjustments based upon a sensor. For example, a sensor may detect the user's eye position and the controller 310 may make physical adjustments such that the light tracks the user's eye. In other examples, the light source 202 and/or associated optics may be configured to produce light in physical dimensions suitable for a range of individuals and/or for an average individual (e.g., physical dimensions of head and location of facial features).

Light exposure, such as may be provided to users by aspects and examples of the systems and methods described herein, may be beneficial to combat fatigue, jetlag, seasonal affective disorder, mood, etc. For example, various forms of light therapy are considered beneficial for various conditions, and a light source associated with a personal audio device (as described herein) may be utilized in certain examples to provide light exposure called for by any particular light therapy. For example, light therapy may help with travel, sleep, meditation, etc. Some light therapy may enhance athletic performance, especially for athletes under a challenging travel schedule, and may be provided by systems and methods herein to thereby improve player wellness.

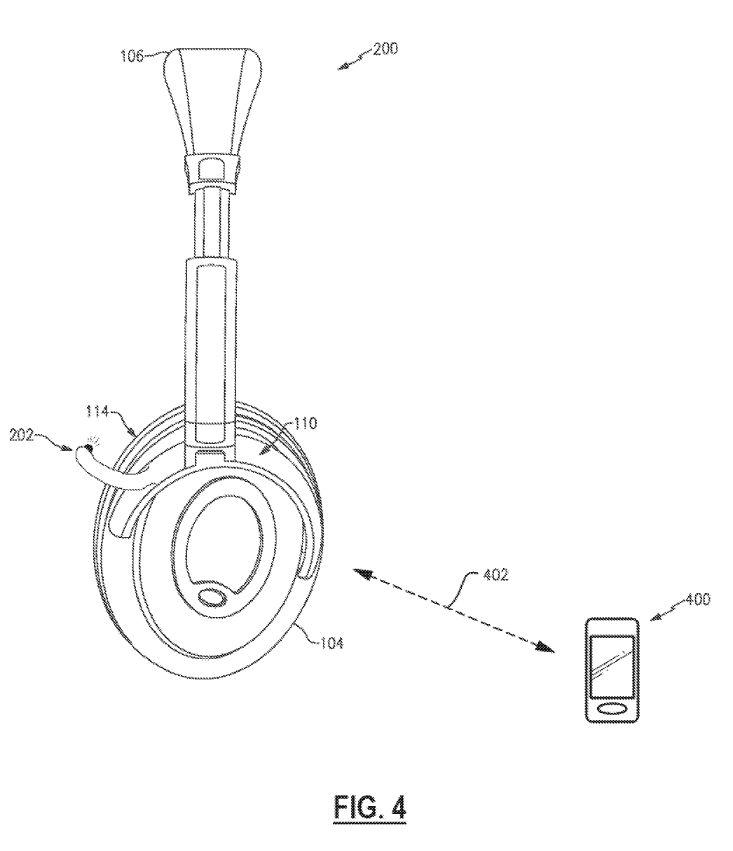

In some examples, light exposure as provided by systems and methods herein may be programmable by a user, or various light programs may be customizable by the user. With reference to FIG. 4, an example user interface is illustrated. In FIG. 4, the headphones 200 (see FIG. 2) include an interface 314 (see FIG. 3) that allows a device 400, such as a smart phone or other user device, to have a wireless communication link 402 with the headphones 200. Accordingly, in various examples, user prompts, screens, and graphical interfaces, etc., may be provided on the device 400 for user control of one or more light programs, that may be stored in a memory, which may be referenced by (or may instruct) the processor 312 (see FIG. 3) in controlling the light source 202. In some examples, a user may be able to directly control the light source 202 (e.g., via the processor 312). In some examples, a number of light programs may be selectable by the user, and/or a light program may include user configurable features, which may include light intensity, color, scheduling, etc. For example, a user may configure details of a desired sleep schedule, which are used by a light program to determine when to turn the light source on and off, what wavelengths or waveband the light source should provide, and intensity of the light provided by the light source. In some examples, the user may configure details of a travel itinerary, and a light program may determine control characteristics of the light source based upon the travel itinerary details and, in some examples, the time of day. In some examples, a user may select a light program based upon a desired effect. For instance, one light program may be intended to encourage wakefulness and/or energy, while another light program may be intended to encourage sleep. In some examples, a user may select any of a number of light control characteristics directly, to establish desired mood lighting, for instance.

In various examples, a light source 202 may be configurable by the user for additional purposes. For example, the user may physically re-adjust the light source 202 to direct light away from the user, such as a reading lamp, headlamp, safety light while walking at night, etc, for example, by bending protruding member 204. In some examples, the user may be provided other options, e.g., via a user interface on the device 400, such as for entertainment uses. For example, a light program may cause the light source 202 to adjust the light in response to music (e.g., via a microphone sensor), or to have a strobe effect. In some examples, the light source 202 may be capable of providing a broader and/or brighter illumination of the user's face so that it may be seen, e.g., by camera or video equipment, by other individuals in the vicinity, etc.

In various examples, any of the functions or methods, and any components of systems (e.g., the controller 310), described herein may be implemented or carried out in a digital signal processor (DSP), a microprocessor, a logic controller, logic circuits, and the like, or any combination of these, and may include analog and/or digital circuit components and/or other components with respect to any particular implementation. Functions and components disclosed herein may operate in the digital domain and certain examples include analog-to-digital conversion (ADC) of analog signals provided, e.g., by microphones, despite the lack of illustration of ADC's in the various figures. Any suitable hardware and/or software, including firmware and the like, may be configured to carry out or implement components of the aspects and examples disclosed herein, and various implementations of aspects and examples may include components and/or functionality in addition to those disclosed.

Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

It is to be appreciated that examples of the methods and apparatuses discussed herein are not limited in application to the details of construction and the arrangement of components set forth in the following description or illustrated in the accompanying drawings. The methods and apparatuses are capable of implementation in other examples and of being practiced or of being carried out in various ways. Examples of specific implementations are provided herein for illustrative purposes only and are not intended to be limiting. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having described above several aspects of at least one example, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. An audio device, comprising:
a structure configured to be worn on a user's head;
an acoustic driver coupled to the structure and configured to convert an audio signal into an acoustic signal in proximity to an ear of the user;
an optical light source coupled to the structure and configured to direct optical energy toward an eye of the user; and
an interface coupled to the light source and configured to control at least one of a color selection, a luminance, or a duration of the optical energy.

2. The device of claim 1 further comprising a light pipe coupled to the light source and configured to deliver the optical energy from the light source to the eye of the user.

3. The device of claim 1 wherein the light source includes a multi-color light emitting device configurable to produce one of a plurality of colors.

4. The device of claim 1 further comprising a sensor coupled to the interface and configured to sense an aspect of the user's physiology, the interface being configured to control the light source based at least in part upon the aspect of the user's physiology.

5. The device of claim 4 wherein the aspect of the user's physiology includes at least one of a heart rate, a respiration rate, a respiration difficulty, an exertion level, a muscle activity, or a blood component level.

6. The device of claim 1 further comprising an ambient light sensor, the light source being configured to adjust the optical energy based at least in part upon a signal from the ambient light sensor.

7. The device of claim 1 further comprising an adjustable component configured to allow user-selectable delivery of the optical energy to a location other than the eye of the user.

8. The device of claim 1 wherein the optical light source is configured to provide optical energy into the eye of the user via a peripheral area of the eye of the user.

9. The device of claim 1 wherein the optical light source is configured to provide optical energy into the eye of the user by illuminating a portion of skin near the eye of the user.

10. A headphone system, comprising:
a structure configured to be worn on a user's head;
an earpiece coupled to the structure;
an acoustic driver coupled to the earpiece and configured to render an audio signal;
an optical light source coupled to one of the structure or the earpiece and configured to direct optical energy toward an eye of the user; and
an interface coupled to the light source and configured to control at least one of a color selection, a luminance, or a duration of the optical energy.

11. The headphone system of claim 10 further comprising a light pipe coupled to the light source and configured to deliver the optical energy from the light source to the eye of the user.

12. The headphone system of claim 10 wherein the light source includes a multi-color light emitting device configurable to produce one of a plurality of colors.

13. The headphone system of claim 10 further comprising a sensor coupled to the interface and configured to sense an aspect of the user's physiology, the interface being configured to control the light source based at least in part upon the aspect of the user's physiology.

14. The headphone system of claim 13 wherein the aspect of the user's physiology includes at least one of a heart rate, a respiration rate, a respiration difficulty, an exertion level, a muscle activity, or a blood component level.

15. The headphone system of claim 10 further comprising an ambient light sensor, the light source being configured to adjust the optical energy based at least in part upon a signal from the ambient light sensor.

16. The headphone system of claim 10 further comprising an adjustable component configured to allow user-selectable delivery of the optical energy to a location other than the eye of the user.

17. The headphone system of claim 10 wherein the optical light source is configured to provide optical energy into the eye of the user via a peripheral area of the eye of the user.

18. The headphone system of claim 10 wherein the optical light source is configured to provide optical energy into the eye of the user by illuminating a portion of skin near the eye of the user.
